(19) United States
(12) Patent Application Publication
(10) Pub. No.: US 2008/0115600 A1
(43) Pub. Date: May 22, 2008

(54) Method and Apparatus for the Mobile Pretratment and Analysis of Catalyst Containing Precious Metals

(76) Inventors: Christian Hageluken, Hanau-Grossauheim (DE); Karlheinz Weitzel, Langenselbold (DE)

Correspondence Address: KALOW & SPRINGUT LLP, 488 MADISON AVENUE, 19TH FLOOR, NEW YORK, NY, 10022, US

(21) Appl. No.: 11/660,003

(22) PCT Filed: Aug. 8, 2005
(86) PCT No.: PCT/EP05/08575

§ 371 Date: Feb. 1, 2008

(30) Foreign Application Priority Data
Aug. 10, 2004 (EP) 04018930.0

(51) Int. Cl. G01N 33/20

(52) U.S. Cl. 73/866

(57) ABSTRACT

Method of determining the precious metal content of catalysts comprising a catalyst housing and a ceramic material containing catalyst material containing precious metal, which comprises the steps (a) provision of a mobile apparatus which comprises a facility for comminuting the ceramic material, a facility for weighing the ceramic material and a facility for taking a sample of the comminuted ceramic material, with the mobile apparatus having a compact construction and being easy to transport, (b) decanning of the catalyst to be analysed by separating the ceramic material from the catalyst housing, (c) comminution and weighing of the ceramic material, (d) taking of a representative sample of the comminuted ceramic material and (e) analysis of the sample to determine the precious metal content of the catalyst. A specific apparatus for the mobile pretreatment and analysis of precious metal containg catalysts is also disclosed.

Catalysts containing precious metals, in particular those on ceramic supports, for example automobile exhaust catalysts and offgas purification catalysts from industrial plants, are being obtained in rapidly increasing amounts as scrap material. The largest part of these is made up of catalysts from deregistered and scrapped motor vehicles. Thus, even in the year 2002, about 60% of the 3.2 million passenger cars deregistered in Germany were equipped with a catalyst containing precious metals, and the proportion is expected to rise to about 100% by the year 2010. In Western Europe, more than 5 million catalysts per year are expected from scrap vehicles by the year 2010. Considerable amounts of the precious metals platinum, palladium and rhodium are present in bound form in these catalysts and the recycling of these is of great economic and ecological importance. Examples of precious metal loadings and values contents of some current automotive catalysts having different precious metal ratios are shown in Table 1, with the precious metal (p.m.) loading being reported in grams per litre of catalyst volume and a price of 19.60 C=/g for platinum (Pt), 5.60 C=/g for palladium (Pd) and 14 C=/g for rhodium (Rh) being assumed.

TABLE 1

| | | Total |
|---|---|---|
| | | intrinsic |
| p.m. | | value |
| loading | | contained |
| g/l | Pt:Pd:Rh | ε/kg |
| 0.7 | 0:3:1 | 8.30 |
| 2.8 | 1:6:1 | 36.30 |
| 2.8 | 5:0:1 | 80.50 |
| 7.1 | 1:15:1 | 74.60 |

An automotive catalyst comprises a metallic or typically ceramic support material whose surface has been coated with a catalytically active “washcoat” in which the precious metals platinum and/or palladium and/or rhodium are present. The coated ceramic catalyst support is typically enveloped by a mineral fibre mat (known as an expanding mat) and installed in a steel housing which is in turn part of the exhaust system of a vehicle.

Until about 1993, the precious metal loading of such catalysts in Europe was relatively uniform with a typical loading of from 1.1 to 1.7 g of precious metal per litre of catalyst volume and a platinum:rhodium ratio of typically 5:1 for vehicles having four-stroke engines. Diesel vehicles were only rarely equipped with catalysts up to this point in time. The loading for diesel vehicles was typically 1.5 g of platinum per litre of catalyst volume. A person skilled in the art was therefore able to conclude the approximate precious metal content of a catalyst from the size of the catalyst and the vehicle type and obtain a good estimate of the recycling value or purchase price of the used catalyst therefrom. Accordingly, the following structures have become established in the recycling chain for catalysts: an automobile recycler sells used catalysts at piece prices (as removed from the motor vehicle) to a buyer. The buyer accumulates used catalysts and sells these to a catalyst decanning (=dismantling) company. The catalyst dismantling company (the decanner) separates the catalyst support containing precious metals from the steel housing by mechanical means, generally hydraulic shears, and collects the ceramic up to amounts of 1000 kg or more. In some cases, the buyer maintains his own decanning plant. The decanner delivers the catalyst ceramic containing precious metals to a precious metals refinery in which comminution, disintegrating or milling, homogenization and representative sampling of the material and subsequently recovery of the precious metals is carried out. Invoicing between the decanning company and the precious metals refinery is done on the basis of the precious metal contents determined by analysis of the sample.

DE 199 42 519 C1 discloses a method for wet chemical determination of the precious metal content of automotive catalysts.

DE 199 20 868 A1 describes a method for separating the ceramic automotive catalysts from the steel housing.

DE 38 03 804 A1 teaches a process for comminuting ceramic catalyst substrates, preferably by a hammer mill.

These references do not disclose a mobile apparatus for the pretreatment and analysis of precious metal containing automotive catalysts.

Since the beginning of the 1990s, the variety of catalyst types in Europe has increased considerably, and these catalysts are increasingly going into recycling circuits, both now and in future years. In the case of catalysts for four-stroke engines, the traditional Pt:Rh ratio of 5:1 with 1.5 g/l of precious metals has been replaced by catalyst coatings containing not only platinum and rhodium but also palladium (Pd), sometimes in dominant amounts (cf. Table 1). Diesel vehicles, too, have increasingly been fitted with catalysts. However, these generally contain only platinum. Overall, both the ratio of Pt, Pd, Rh to one another and the precious metal loading in automobile catalysts have since then fluctuated within a very wide range (cf. Table 1). The absolute and relative prices of these precious metals and thus the intrinsic value of the catalysts are likewise subject to extreme fluctuations. As can be seen from Table 1, the intrinsic value in the same catalyst volume can vary by a factor of up to 10 depending on the precious metal loading. Since the variations in the precious metal contents also occur within one make of automobile, even a person skilled in the art can no longer see at a glance what approximate precious metal content the catalytic converter (“cat converter”) has and how the purchase price is to be set accordingly.

The above-described structures of the recycling chain are no longer appropriate for the more recent catalysts and have logistic, technical and commercial disadvantages which will be briefly summarized below.

Between purchase of the catalyst by the automobile recycler and analysis of the actual precious metal (p.m.) contents, there are generally large periods of time of generally at least 2 months, but often also 6 months and more. Since both the buyer and the decanner accumulates catalysts, cat converters and cat ceramic, it is generally not possible for an p.m. content to be assigned to a quantity of converters taken over by the automobile recycler X, but instead it is only possible to determine the average content of a collection from many suppliers. Neither sellers nor buyers can thus discover in an individual case whether they have paid or received a fair price for their goods. The logistic and commercial practices of catalyst recycling which have prevailed hitherto will no longer be able to be continued with the greatly increasing proportion of the above-described new catalysts having widely varying precious metal loadings in future years without sellers and buyers being exposed to considerable uncertainties and risks.

Apart from the abovementioned economic disadvantages, there are also considerable technical disadvantages in the existing procedures. In addition to losses of precious metal, considerable environmental risks can also occur. Due to transport and frequent transfer of the cat converters before decanning results in dust and thus an average of about 5% of precious metal being lost. Cat converters are classified as hazardous waste requiring particular monitoring (“bü” under German regulations) because of the ceramic fibres of the expanding mat present therein. The current recycling chain requires considerable transport of this “bü” waste. This entails the risk of ceramic fibres being released due to frequent transfer and inappropriate handling.

The dismantling (decanning) of the catalyst, i.e. the removal of the catalyst ceramic containing precious metal is at present not always carried out properly using the necessary safety equipment. If decanning is carried out unprofessionally, there is not only a risk of injury to workers but also the danger of dust/precious metal losses and emission of ceramic fibre dusts which are hazardous to health, especially if dust extraction is absent or incorrectly dimensioned.

It is an object of the present invention to provide a method, an apparatus and the use of this apparatus in order to solve the above mentioned problems. A further object is to improve the precious metals yield in recycling of the catalysts by means of the method of the invention and the apparatus of the invention and at the same time minimize the environmental risks.

A further object of the present invention is to provide a method of determining the precious metal content of the catalysts on site.

These objects are achieved by the features of the claims. Advantageous embodiments of the invention form the subject matter of the dependent claims.

The mobile apparatus of the invention for pretreatment and determining the precious metal content of catalysts comprising a catalyst housing and a ceramic material containing catalyst material containing precious metal comprises a comminution facility for comminuting the ceramic material, a facility for weighing the ceramic material and a facility for taking a sample of the comminuted ceramic material which is representative of the catalyst. Furthermore, the mobile apparatus has a compact construction and is easy to transport. The apparatus preferably also has a facility for decanning the catalyst to be analysed. The mobile apparatus preferably has a casing or a device for partial encapsulation in order to encapsulate the apparatus and isolate it from the environment in an airtight and/or dusttight manner, by which means the emission of environmentally harmful dusts can be prevented and safe and environmentally friendly operation is made possible. In a preferred embodiment, the encapsulation has at least one dust extraction facility by means of which the gases and/or dusts can be extracted effectively and can subsequently optionally be filtered and collected.

Owing to the embodiment as a mobile apparatus according to the invention and the modular construction of the apparatus according to the invention, a plurality of process steps for the pretreatment of catalysts can be carried out at one place, as a result of which precious metal losses can be minimized. A further technical advantage of the apparatus of the invention and the process of the invention is that the individual process steps can be matched individually and thus always optimally to the conditions prevailing at the particular site. The provision of the various units of the decanning facility, comminution facility, the weighing facility, the sampling facility and optionally the analytical facility enables a continuous process which allows optimized precious metal recycling to be achieved.

The apparatus of the present invention preferably has a modular construction, so that specific parts, like pre-treatment modules and determining modules can be individually or commonly arranged. In particular, a decanning or dismantling facility, a comminution or disintegrating facility for comminuting the ceramic material, a facility for weighing the ceramic material, a facility for taking a sample of the comminuted ceramic material which is representative of the catalyst and a facility for analysing a sample to determine the precious metal content of the catalyst may be provided as individual modules which are adapted to be arranged either alone or in combination with a mobile apparatus having a compact construction and being easy to transport. Further modules may be combined with one or more of the above modules such as a casing, an extraction facility preferably a dust extraction facility and/or a waste module for collecting remaining waste or recycling materials. Preferably a facility or several facilities are arranged on a trailer or several trailers. The present invention is further advantageous in that the decanning module may be provided on one trailer and other modules may be provided on at least one separate trailer.

Therefore, in case an automobile recycler carries out the decanning step himself, only the trailer with the other modules is needed for the method of pretreating and determining according to the present invention.

Providing a plurality of individual modules has the advantage that the method of pretreatment and determining the precious metal content of catalyst may be carried out in a coupled manner on the mobile apparatus, i.e. at one place instead of carrying out the plurality of process steps over various locations. Furthermore, providing a plurality of modules is advantageous in that the individual modules may be customized such that coupled method steps can be carried out in a smooth and continuous manner. In other words, the efficiency or working speed of the individual modules can be matched. Thus, the steel from the steel housing can be left at the one place and the analysing of the sample to determine the precious metal content of the catalyst can be carried out at the same place which reduces process costs and metal losses can be minimized.

Preferably, the modules comprise common interfaces for energy supply, water supply and/or waste water disposal such that the pretreatment and the analysis may be carried out at any location with minimum installation efforts. However, according to a further aspect of the present invention, the mobile apparatus may also comprise a power supply module, a module tank and/or a container for sewage such that the apparatus is not necessarily dependent on the above means.

An automobile recycler usually sells used catalysts piecewise. As first working step, the decanning of the catalyst has to be carried out. In the following, the term “decanning” refers to a process step by means of which a cat ceramic containing precious metals is removed from the housing. Professional removal of the ceramic cat monoliths from the steel housing is generally carried out manually with the aid of hydraulic shears or presses. In a preceding step, exhaust pipes which may still be present are separated or cut off from the cat converter (monolith with steel housing). The separation of the ceramic monolith containing precious metals from the steel housing is then carried out by means of hydraulic shears optimized specifically for catalyst decanning. In general, the converter is parted radially by means of a first cut, with the ceramic monolith usually breaking and fine fractions being formed. Since the sheet metal housing is squeezed together and the ceramic is enclosed as a result of this first cut, a second cut after rotation through 90° is generally necessary to open the housing again. In both cuts, the converter is introduced manually and held by an employee. The ceramic containing precious metal is subsequently emptied from the two parts of the converter into suitable containers, preferably 200 l drums, and the remaining steel fraction is separately collected or classified according to various grades of steel. Thus, providing the apparatus of the present invention with a module for decanning has the advantage that the steel from the steel housing can be left by the automobile recycler.

Since construction types, geometries and sizes of the cat converters can vary greatly, non-manual, automated decanning is uneconomical and technically difficult to achieve. The quality and completeness of the separation of ceramic and steel fraction depends essentially on the following factors: the engineering design and equipping of the hydraulic shears/press; the auxiliary equipment or the periphery; the arrangement and ergonomic configuration of the working area and the experience and capabilities of the employee. Owing to the modular construction of the present invention, it is possible to optimize the above factors or match them to the circumstances of the recycler or seller.

In the process step of decanning, many boundary conditions generally have to be taken into account. The apparatus should have installed safety devices such as an emergency off. To operate the apparatus, protective clothing usually has to be worn to provide the employee with optimal protection during decanning, in particular protection from injury by cutting tools or splitters. Furthermore, effective dust extraction facilities should be installed at the place of cutting, on the emptying containers and/or in the general region of the apparatus. This serves to protect the employee from ceramic fibres which may be hazardous to health from mineral expanding mats of the catalyst and to minimize precious metal losses through dusts. Furthermore, dusts or fibres and/or gases which arise can be filtered or purified by means of suitable extraction facilities and, if appropriate, collected in separate containers, as a result of which an environmentally friendly process is ensured. A high throughput and long life of the plant, in particular of the cutting tools, is desirable. Especially in the case of the mobile apparatus of the invention for the pretreatment of catalysts, a compact construction, good transportability and good cleaning opportunities are also desirable in order to avoid cross-contamination and sample falsification between different catalyst suppliers. The apparatus should also preferably be able to be operated by a single person.

In present-day practice, i.e. in the distribution of a plurality of process steps over various localities, methods which are problematical in terms of safety and cause relatively high precious metal losses are sometimes still employed. These include decanning without dust extraction or inadequately dimensioned extraction, opening of converters using welding torches, bow saws or cutters and also careless handling of converters, for example repeated transfer with dropping and impacts, as a result of which considerable dust losses accompanied by corresponding precious metal losses can occur. According to a current study, higher total precious metal losses can be assumed here than in the case of melting of the catalyst ceramic and refining of the precious metals. Total shredding of cat converters with subsequent separation of ceramic dust and steel likewise appears to be unsuitable. Very much more ceramic dust is generated in the shredding process than in decanning by means of hydraulic shears/presses and this leads to great problems in practical operation.

In a preferred embodiment of the method or apparatus of the invention, the ceramic is comminuted or milled and, if appropriate, homogenized in a comminution step after decanning so that a representative sample can be taken therefrom. The cat ceramic which has been separated off is preferably comminuted in a (screen) ball mill to a particle size of less than about 2 mm, preferably to a size in the range of about 0.5-2 mm. Other suitable milling apparatuses such as roll mills, cutting mills, impact mills, hammer mills, jaw crushers, etc, can likewise be used. As ball mills, it is possible to use suitable, generally cylindrical, standard apparatuses using milling media made of steel. The closed construction of the milling container avoids dust emissions, and in addition the ball mill should also be provided with dust extraction. The rotation of the milling container simultaneously produces mixing and homogenization of the material being milled and thus creates the prerequisites for subsequent sampling. In the preferred embodiment as screen ball mill, fine material is discharged when its size is below the screen opening. As a result, overmilling of the material being milled is prevented. The output from the ball mill preferably goes directly into an automatic sample divider, preferably a rotary tube divider, by means of which a representative raw sample of a batch of cat ceramic is obtained. The (screen) ball mill of the apparatus of the invention should have a compact construction and in addition be transportable. The throughput of the comminution facility should be matched to the preceding decanning facility, be easy to clean and have a modular construction for optimized material flow.

During the course of decanning, comminution and later sampling by means of the mobile apparatus of the invention, it is important to be able to determine and record the exact weights of the ceramic fraction containing precious metals on site. The cat ceramic present can be weighed by means of a weighing facility either before and/or after comminution, so that the weight can be documented in a suitable form. The weighing facility is preferably likewise a modular constituent of the apparatus of the invention in order to ensure very optimal material flow and transparent documentation. For this reason, this weighing facility is, in a further preferred embodiment, linked to a record printer and provided with an EDP interface.

Furthermore, the mobile apparatus is preferably equipped with an analytical facility for precious metals. A suitable measurement apparatus, preferably X-ray fluorescence analysis (XRF), preferably energy-dispersive XRF, can be used for this purpose. Suitable methods are provided for sample preparation, for example conversion of the raw sample into a representative measurement sample. In one embodiment, the raw sample is comminuted or milled to 0.2-0.3 mm, preferably in a vibratory disc mill, and divided into measurement (laboratory) samples, preferably in a riffle divider or a rotary tube divider.

In particular, the apparatus can be used not only for automobile catalysts but also for other suitable materials, in particular those containing precious metals. Examples are other materials having a ceramic matrix, e.g. bulk catalysts, fixed bed catalysts or pellet catalysts for the chemical industry and catalytic after-combustion, particulate refractory bricks containing precious metals, e.g. from the glass industry, or ceramic residues from the electronics industry. The sample which has been taken is preferably subjected to on-site analysis to determine the approximate content of the precious metals. In a preferred embodiment, the apparatus has a modular construction which allows flexible replacement of individual facilities and flexible use of the individual facilities. The process steps of decanning, comminution or milling and/or sampling and analysis can be coupled or can occur independently of one another. However, the individual process steps preferably proceed continuously.

The mobile apparatus can in particular embodiments dispense with integrated decanning. In this case, the cat ceramic would be decanned independently of the apparatus of the invention, for example in a stationary decanning facility, and subsequently fed to the mobile apparatus, comminuted, sampled and weighed. Analysis of the sample taken can be carried out on the mobile apparatus or else at a later point in time in a suitable stationary laboratory. The mobility of the apparatus of the invention can be achieved by mounting or carrying the module on a platform of a vehicle, in a vehicle itself or on a trailer, preferably with common interfaces for energy supply, water etc. This has the advantage that the apparatus according to the present invention is mobile and can be driven to different sites.

Compared to apparatuses known from the prior art, the mobile apparatus of the invention offers the decisive advantage that the precious metal contents of the catalysts can be determined as early as at the point at which they are obtained, e.g. at the automobile recycler. In addition, the on-site pretreatment ensures reliable assignment of the specific quality and minimizes precious metal losses. This makes possible a high transparency for seller and buyer and detailed traceable recycling routes. Furthermore, the mobile apparatus ensures, owing to its safety precautions, professional handling of the materials to be recycled which is safe both for the operator and for the environment. The preferred embodiment of an encased apparatus avoids emissions of dust fibres/ceramic fibres which are hazardous to health. Owing to the fact that many process steps are carried out completely, preferably continuously, at the place where the material is obtained, precious metal losses during transport and transfer of cat converters is avoided. Furthermore, carrying out many process steps at one location minimizes transport flows.

The method of the invention or the operation of the apparatus of the invention are illustrated below with the aid of a specific operational example. However, the invention is not restricted to the following example and numerous modifications are conceivable within the scope of the invention.

### EXAMPLE

An automobile recycler (seller) has 150 automotive catalysts which he wishes to sell for recycling.

The mobile apparatus of the invention comprising
a facility for decanning,
a facility for comminution,
a facility for homogenization with sampling,
a weighing facility and
an analytical facility with X-ray fluorescence instrument is brought to the premises of the automobile recycler. The catalysts have not yet been decanned, but the seller has previously cut off the exhaust pipes. After joint assessment of the catalysts, these are decanned by means of the facility on the mobile apparatus and the number is confirmed and documented. The dust formed during decanning is extracted, collected and later added to the dusts obtained during milling. The automobile recycler takes the steel housing of the catalytic converter which has been separated off during decanning and markets this directly with similar iron fractions obtained in his operation. The catalyst ceramic obtained on decanning is placed in a feeder container (hopper) from where it is passed via a metering device directly to the milling facility. The milled material is discharged from the milling facility via an automatic sample divider from which a substream of, for example, 2% is continuously taken off and separately measured (“raw sample 1”). The main stream goes directly into 200 l drums which, after they have been filled, are in each case closed, sealed and marked with an identification number. In the case of the number of catalysts specified in the example, about 150 kg of milled catalyst ceramic, which is generally sufficient for a 200 l drum, can be expected. The dusts extracted during milling are combined with the decanning dusts, homogenized (in a homogenization facility, e.g. in a tumble mixer) and a representative sample (raw sample 2) is taken therefrom. All fractions obtained, i.e. the main fraction of the milled ceramic, raw sample 1, dust fraction and raw sample 2, are weighed (in an automatic balance provided with a record printer and EDP interface) and recorded in a document together with the number of catalysts determined during decanning. The raw samples 1 and 2 are processed further by known methods to give a laboratory sample and are analysed to determine their precious metal content by means of an X-ray fluorescence instrument (XRF) installed on the apparatus. The precious metal contents of samples 1 and 2 are likewise recorded in the document, and the document is signed by both parties after conclusion of the work. Invoicing between seller and buyer is effected on the basis of the weights and analytical values recorded in the document and the conditions agreed beforehand.

The sealed drums are stored by the automobile recycler and at a later point in time are taken over by a transport contractor and brought to the precious metals refinery.

All relevant data are recorded, monitored and balanced electronically.

The procedure described ensures that no dust emissions with their associated health risks and precious metal losses occur. All steps are carried out with great transparency, in closely matched processes and with great professionalism.

1. Method of pretreatment and determining precious metal content of a catalyst that comprises a catalyst housing and a ceramic material which comprises a catalyst material containing precious metal, the method comprising the steps of:
(a) providing a mobile apparatus which comprises a facility for comminuting the ceramic material, a facility for weighing the ceramic material and a facility for taking a sample of the comminuted ceramic material, wherein the mobile apparatus has a compact construction and is easy to transport,
(b) decanning or dismantling the catalyst to be analyzed by separating the ceramic material from the catalyst housing,
(c) comminuting and weighing the ceramic material,
(d) taking a representative sample of the comminuted ceramic material and
(e) analyzing the sample to determine the precious metal content of the catalyst,
with at least steps (c) and (d) being carried out in a coupled fashion on the mobile apparatus.

2. Method according to claim 1, wherein the process steps (b) to (e) proceed continuously in succession.

3. Method according to claim 1, wherein the mobile apparatus has a casing to at least partially encapsulate the mobile apparatus and isolate it from the environment.

4. Method according to claim 3, wherein the casing has at least one dust extraction facility.

5. Method according to claim 1, wherein the ceramic material is comminuted by milling and subsequently homogenized in step (c).

6. Method according to claim 1, wherein step (e) is carried out independently in a suitable stationary laboratory.

7. Method according to claim 1, wherein the steps (b) to (d) proceed in a coupled fashion.

8. Method according to claim 1, wherein step (b) is carried out in a stationary decanning facility.

9. Method according to claim 1, wherein the steps (b) to (d) are carried out in a mobile apparatus.

10. Method according to claim 1, wherein a plurality of catalysts are decanned and their ceramic material is comminuted in order to determine the total precious metal content of the plurality of catalysts.

11. (canceled)

12. Mobile apparatus for pretreatment and determining precious metal content of a catalyst that comprises a catalyst housing and a ceramic material which comprises a catalyst material containing precious metal, the apparatus comprising a comminution facility for comminuting the ceramic material, a facility for weighing the ceramic material and a facility for taking a sample of the comminuted ceramic material which is representative of the catalyst, wherein the mobile apparatus has a compact construction and is easy to transport.

13. Apparatus according to claim 12, wherein the mobile apparatus has a casing to encapsulate the mobile apparatus and isolate it from the environment.

14. Apparatus according to claim 13, wherein the casing has at least one dust extraction facility.

15. Apparatus according to claim 12 which further comprises an analytical facility for analyzing the sample so as to determine the precious metal content of the catalyst.

16. Apparatus according to claim 12 which further comprises a decanning facility for separating the ceramic material from the catalyst housing.

17. Apparatus according to claim 12, wherein the comminution facility has a facility for milling and homogenizing the ceramic material.

18. Apparatus according to claim 16, wherein at least one of the comminution facility, the facility for weighing, the facility for taking a sample, or the decanning facility is an individual module which can be arranged individually or combined with a mobile construction.

19. Apparatus according to claim 18, wherein one or a plurality of modules, preferably the decanning facility, is/are arranged on at least one trailer.

20. A method for determining the value of at least one used catalyst on the basis of the precious metal content of the at least one used catalyst, the method comprising using the apparatus of claim 12.

21. A method of pretreatment and determining precious metal content of a catalyst which comprises using the apparatus according to claim 12.
